(19) United States
(12) Patent Application Publication
(10) Pub. No.: US 2026/0035852 A1
(43) Pub. Date: Feb. 5, 2026

(54) METHOD FOR TREATING PERSONAL PROTECTIVE EQUIPMENT

(71) Applicant: Cool Clean Technologies, LLC, Eagan, MN (US)

(72) Inventors: Nelson W. Sorbo, Savage, MN (US); Jon R. Turner, Minneapolis, MN (US); Jon Wikstrom, St. Paul, MN (US)

(21) Appl. No.: 19/354,258

(22) Filed: Oct. 9, 2025

Related U.S. Application Data
(62) Division of application No. 17/390,419, filed on Jul. 30, 2021.
(60) Provisional application No. 63/058,861, filed on Jul. 30, 2020.

(51) Int. Cl. D06F 43/00; A47L 23/02

(52) U.S. Cl. CPC D06F 43/007; A47L 23/02

(57) ABSTRACT

A method for cleaning and decontaminating apparel includes placing the apparel into a pressurizable cleaning vessel. The cleaning vessel is filled with a solvent comprising a mixture selected from at least one of propylene glycol ether, water, organic acid and carbon dioxide. After the apparel has been in contact with the solvent for a first selected period of time, carbon dioxide gas under pressure is introduced into the cleaning vessel to carbonate the solvent. After appropriate agitation, at least a portion of the solvent is then removed from the cleaning vessel while under pressure. A rinsing solution, comprising liquid carbon dioxide and alcohol, is then introduced into the cleaning vessel under pressure. After a third selected period of time, the rinsing solution is removed from the cleaning vessel under pressure. Finally, the cleaning vessel is depressurized and the apparel is removed from the cleaning vessel.

### CROSS-REFERENCE TO RELATED APPLICATIONS

The present application claims benefit of U.S. Provisional Application No. 63/058,861 filed Jul. 30, 2020, the contents of which are each incorporated herein in its entirety by this reference.

### BACKGROUND OF THE INVENTION

The present invention is directed to the field of cleaning and disinfecting apparel. More particularly, the present invention is directed at cleaning and disinfecting personal protective equipment (“PPE”) as is used by firefighters, emergency medical workers and health care workers. PPE includes, but is not limited to, protective clothing, helmets, goggles, pants, coats, gloves, hoods, boots and interface elements, or other garments or equipment designed to protect the wearer's body from injury or infection. The hazards addressed by protective equipment include physical, electrical, heat, chemicals, biohazards, and airborne particulate matter. PPE may be worn for job-related occupational safety and health purposes, as well as for sports and other recreational activities. PPE may include “protective clothing” as applied to traditional categories of clothing, and “protective gear” as applied to items such as pads, guards, shields, or masks, and others. Systems utilizing carbon dioxide have been used to clean a wide variety of articles, including garments, textiles, hardware, aerospace components and medical devices and articles. In some cases, these goals are achieved by using carbon dioxide at selected pressures and temperatures, or with additives in a co-solvent system.

While the use of a cleaning solvent followed by a liquid carbon dioxide rinse is well understood for cleaning garments and other hardware, the rate of cleaning, including removal of heavy metals, or disinfecting of a wide variety of bacteria, viruses and other organisms generally fell below that as required by certain industry standards. Such standards include those as set by U.S. Environmental Protection Agency standards for per- and polyfluoroalkyl substances (PFAS), National Fire Protection Association (NFPA) 1851, and other Occupational Safety and Health Administration (OSHA) standards. This also includes the need for decontaminating PPE from human corona virus.

There therefore exists a need to provide a cleaning system which can effectively and efficiently clean and decontaminate PPE to meet varying standards without adversely affecting the PPE itself for prolonged life and longevity of the material.

### SUMMARY OF THE INVENTION

The present invention enables carbonated solvents to contact articles of apparel for improved cleaning, decontaminating and disinfecting performance. In addition to enhanced cleaning using this method, this process has been shown to disinfect selected organisms by at least Log 4 (i.e., 99.99% eradication of selected organisms). Furthermore, tests conducted on human corona virus have shown viral inactivation at the level of Log 4. As such, the use of the processes and system of the present invention will be particularly beneficial to cleaning and disinfecting PPE, as well as other articles requiring both cleaning and disinfection.

The present invention describes processes and systems for cleaning, decontamination and disinfecting articles in an apparatus utilizing dense phase carbon dioxide and selective cleaning and disinfecting agents. Two process embodiments can be used to accomplish this objective. The first process embodiment is accomplished by exposing the articles to be processed first to a solvent which both cleans and disinfects, followed by multiple liquid carbon dioxide rinse cycles which are used to remove the residual solvent and residues from the articles. The second process embodiment exposes the articles to be processed to various mixtures of liquid carbon dioxide and customized detergents, followed by a liquid carbon dioxide rinse. In either embodiment, the articles are removed from the cleaning vessel cleaned, disinfected and dry. Hazardous residues are then removed from the cleaning agents, and the carbon dioxide is distilled and reused in the process.

### BRIEF DESCRIPTION OF DRAWINGS

The following FIGURES are used herein in conjunction with the written description to assist in understanding the invention. The Figures are as follows:

FIG. 1 is a flow diagram of the apparatus and method for treating PPE in accordance with the present invention.

### DETAILED DESCRIPTION OF THE INVENTION

The present invention as described herein is directed at systems and methods for cleaning, decontaminating and disinfecting, including the removal of heavy metals and semi volatile organic compounds (“SVOC's”) from apparel and equipment, preferably personal protective equipment (“PPE”). For purposes of this description, by “PPE” it is meant, but is not limited to, equipment worn to minimize exposure to hazards that cause serious injuries and illnesses, including but not limited to pants, coats, gloves, hoods, helmet, boots and interface elements to be cleaned or disinfected. Such items may also be generally referred to simply as apparel.

This present invention includes a first embodiment utilizing a solvent cleaning prewash followed by a liquid carbon dioxide rinse process. The present invention also includes a second embodiment utilizing detergent additives in combination with the liquid carbon dioxide wash of the first embodiment, followed by a liquid carbon dioxide rinse. Preferably, both the first embodiment and the second embodiment can be conducted in the same machine or vessel.

To perform the process of the present invention in connection with the first embodiment, articles of apparel, such as PPE, may be placed in a permeable processing bag designed to confine and protect the articles during the cleaning process while providing intimate contact of the process solution so as to interact with said articles of apparel. The bags may be constructed of non-reactive porous materials such as polyester, a fabric known in the art and used in laundry cleaning operations. The processing bag containing the articles of apparel is inserted into the cleaning vessel whereupon the chamber door is secured. An exemplary pressurizable cleaning vessel for use with the present invention includes that as disclosed in commonly owned U.S. Pat. No. 6,851,148, the contents of which are hereby incorporated by reference. However, it should be recognized that the use of other pressurizable cleaning vessels capable of withstanding the pressures needed are well within the scope of the present invention.

With the bag positioned within the cleaning chamber, an environmentally friendly biodegradable chemical solvent is added to partially fill the cleaning vessel. Preferably, the preferred solvent is selected from the propylene glycol ether family and can be mixed with other cleaning agents and brighteners. Preferred examples of solvent and other cleaning agents include, but are not limited to, between 80% and 100% glycol ether, up to 15% water, up to 5% organic acid and up to 10% carbon dioxide. As the solvent is added, a rotating basket contained within the cleaning vessel rotates about its horizontal axis, generating a ‘lift and splash’ cleaning action. The solvent, along with other cleaning agents, including water, surfactants and acids, is effective in cleaning a wide range of contaminates, both polar and non-polar contaminants, and hazardous compounds. The solvent used in this process mode has been shown to disinfect selected organisms by at least Log 4-6 (i.e., 99.99%-99.9999% bacterial eradication) and inactivate human corona virus to a level of Log 4.

After a period of time, typically between 5 to 15 minutes, gaseous carbon dioxide is injected into the cleaning vessel with increasing pressure. As a result from the increasing pressure, an increase in temperature of solution due to the heat of mixing of the gaseous carbon dioxide and the cleaning solvent occurs. As this happens, the density of the cleaning solvent decreases, whereby the effective liquid level of the cleaning solvent in the cleaning vessel increases, the viscosity of the cleaning solvent decreases, and the solubility of the cleaning solvent decreases. The result of this action modifies the cleaning chemistry by creating a cleaning agent that more effectively penetrates the interior of the articles in the cleaning vessel. This is particularly so in PPE that may be water-resistant or water-proof. Carbonating the solvent decreases the solubility of the cleaning agent, causing some residues to fall out of solution resulting in a more effective filtration process as the solvent is pushed out of the cleaning vessel. Preferable carbonation pressures can range from 200 to 800 psig (1375 to 5500 kPa) but can be as high as 1000 psig (6900 kPa). Under pressure with the carbon dioxide gas, the mixture is agitated about the horizontal axis to enhance mixing of the solvent with the articles to be processed.

To further enhance mixing, the gaseous pressure is decreased, causing carbon dioxide gas to come out of solution and generate in situ convection in and around the articles of apparel being cleaned. This is immediately followed by an increase in gaseous pressure, typically up to 200 psig (1375 kPa) and the process is repeated. This process of increasing and decreasing pressure in cycles, which generally takes between one and two minutes per cycle, is also effective in enhancing the cleaning of articles in the cleaning vessel. During this process of increasing and decreasing pressure, which is generally between 1 and 10 cycles, preferably 4, along with agitation, it has been discovered that the cleaning agent had a demonstrated capability to remove a wide variety of residues from articles. Furthermore, this agent in combination with carbon dioxide has been shown to achieve greater than 4 Log disinfection of vegetative bacteria, and at least a 6 Log disinfection of other bacteria. Further, this process has been shown to achieve a Log 4 viral inactivation level using human corona virus. Hence this process can both clean and disinfect in a single step.

At the conclusion of the wash solvent cleaning step, the carbonated solvent is transferred under pressure from the cleaning vessel, through a filter, and to the solvent still. The process then proceeds to the rinsing cycle. The rinsing cycle, which includes the introduction of liquid carbon dioxide ranging from 95%-100% by mass, with optionally selected rinse additives, including up to 5% alcohol, is designed to remove residual contaminates and cleaning solvent from the articles of apparel, as well as from the interior of the cleaning vessel. Liquid carbon dioxide is added to the pressure vessel and agitated for a period of time to solubilize the residual cleaning solution on the articles and in the cleaning vessel. The mixture is then transferred to the carbon dioxide still. Typical carbon dioxide rinse pressures range from 400-700 psig (2750-4850 kPa), but can go as high as 1000 psig (6900 kPa) and as low as 250 psig (1725 kPa). The rinsing step may be repeated as necessary to achieve the desired removal of cleaning solvent.

Once the rinsing cycle has been completed, any remaining gaseous carbon is removed from the cleaning vessel and the pressure brought back to atmospheric conditions. The cleaning vessel door is then opened and the bags containing the articles of apparel having been processed are removed in a clean, decontaminated, dry and cool state. As part of the waste separation and recycling system, filters and stills are used to segregate waste products from solvent, detergents and carbon dioxide. As spent carbon dioxide-solvent-detergent mixtures are conveyed out of the cleaning vessel, they are passed through a filter, or series of filters, to remove suspended materials from the solution. The mixtures are then transported to the still, where heat is used to boil off liquid carbon dioxide, leaving still bottoms consisting of solvent-detergents and waste products. Gaseous carbon dioxide is conveyed to the heat exchanger in which it is condensed to liquid carbon dioxide which is directed to the carbon dioxide storage tank for reuse. A separate solvent tank or still system may be used to store and purify the pre-wash solvents used in the process.

To perform the process of the present invention in connection with the second embodiment, articles of apparel to be cleaned or disinfected are placed in a similar processing bag as previously described, which is designed to confine and protect the articles of apparel during the cleaning process while providing easy access of the process solution to intimately contact and interact with the articles of apparel. The processing bag(s) containing the articles of apparel is inserted into the cleaning vessel and the door is secured.

Gaseous carbon dioxide is introduced into the cleaning vessel, generally between 300 and 800 psig (2050 to 5500 kPa), preferably at about 600 psig (4100 kPa) followed by the introduction of liquid carbon dioxide. Upon reaching a selected amount or level of liquid carbon dioxide, preferably between 95-100% by weight, one or more selected detergent additives may be injected into the cleaning vessel. Preferable detergent additives include, but are not limited to, up to 2% by weight isoparaffinic detergents with non-ionic and anionic surfactants, up to 2% by weight organic acids, up to 2% by weight terpene, up to 2% by weight alcohol based detergents, up to 2% by weight glycol ether-based additives, up to 2% by weight alcohols, up to 1% by weight non-ionic surfactants, up to 2% by weight water, up to 2% by weight hydrogen peroxide, up to 2% by weight docusate salts, up to 2% by weight ketones, and combinations thereof. Examples of organic acids including, but are not limited to, citric acid, acetic acid and carbonic acid. Preferable carbon dioxide wash pressures range from 400-700 psig (2750-4850 kPa) but can go as high as 1000 psig (6900 kPa) and as low as 250 psig (1725 kPa).

The mixture is agitated about a horizontal axis to enhance mixing of the solvent with the articles to be processed. To further enhance mixing, the gaseous pressure is decreased, generally from 600 to 400 psig (4100 to 2750 kPa) causing the carbon dioxide gas to come out of solution and generate in situ convection in and around the articles of apparel. This is followed by an increase in gaseous pressure which has been found to be effective in enhancing the cleaning of articles in the cleaning vessel. The increase and decreasing of pressure cycle can be repeated. Typical pressures in this pumping ranges can be between 50-200 psig (345-1375 kPa), but can be as high as 400 psig (2750 kPa). At the conclusion of the wash cycle, the carbon dioxide-additive mixture is extracted from the cleaning vessel and fed through a filter, and then to the carbon dioxide still.

Next, a rinse cycle occurs by introducing a rinsing solution to the cleaning vessel. The rinsing solution includes liquid carbon dioxide, preferably between 95% and 100% by weight, being added to the cleaning vessel. The rinsin solution may optionally include other additives, including but not limited to, up to 2% by weight alcohol, up to 1% by weight hydrogen peroxide, and up to 2% by weight water. The rinsing solution removes residual contaminates and cleaning solvent from the articles and from the interior of the cleaning vessel. Upon adding the rinsing solution, the vessel is agitated for a period of time to solubilize the residual cleaning solution on the articles and in the cleaning vessel. The mixture is then drained and fed through the carbon dioxide still. Typical rinse pressures range from 400-700 psig (2750-4850 kPa) but can go as high as 1000 psig (690 kPa) and as low as 250 psig (1725 kPa). The rinse cycle may be repeated as often as necessary to achieve the desired removal of cleaning solvent. Upon completion of the rinse cycle, remaining gaseous carbon dioxide is removed from the cleaning vessel until atmospheric pressure is reached. The cleaning vessel door is opened, and the bags of processed articles are removed in a clean, decontaminated, dry and cool state.

As part of the waste separation and recycling system, filters and stills are used to segregate waste products from solvent/detergents and carbon dioxide. As spent solvent/detergent/carbon dioxide mixtures are conveyed out of the cleaning vessel, they are passed through a filter, or series of filters, to remove suspended materials from the solution. The mixtures are then conveyed to the still, where heat is used to boil off liquid carbon dioxide, leaving still bottoms consisting of solvent/detergents and waste products. Gaseous carbon dioxide is conveyed to the heat exchanger in which it is condensed to liquid carbon dioxide which is directed to the carbon dioxide storage tank for reuse. A separate solvent tank/still system may be used to store and purify the pre-solvents used in the process.

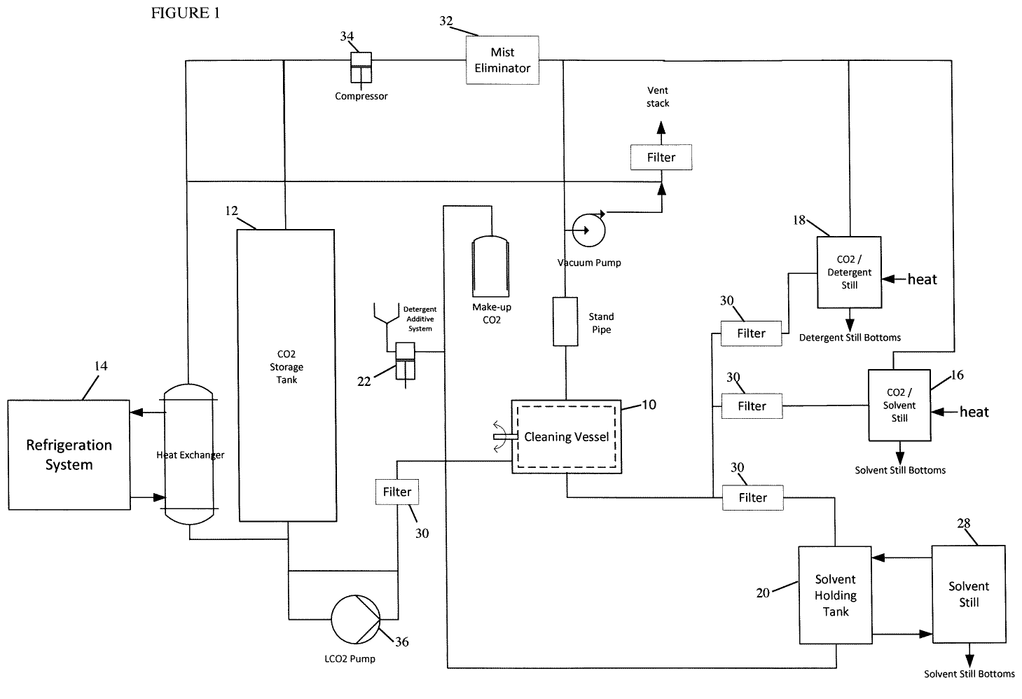

Referring now to FIG. 1, a cleaning vessel is indicated at 10. The cleaning vessel 10 preferably includes a pressure vessel horizontally mounted with a rotating perforated basket and a pressure rating enough to accommodate the process pressures. A carbon dioxide storage tank 12, for holding clean carbon dioxide, connects to the cleaning vessel 10 via conduits and associated piping. A heat exchanger/refrigeration system 14 connects to the storage tank 12, and provides process cooling to condense gaseous carbon dioxide. A carbon dioxide/solvent still 16, preferably a pressure vessel, is designed to hold and vaporize the liquid carbon dioxide in mixtures of liquid carbon dioxide/cleaning solvent/residues, which have been conveyed from the cleaning vessel 10 from the rinse step. A carbon dioxide/detergent still 18, preferably a pressure vessel, is designed to hold and vaporize the liquid carbon dioxide in mixtures of liquid carbon dioxide/detergent additives/residues which have been conveyed from the cleaning vessel 10 from the wash and rinse steps. A cleaning solvent holding tank 20, preferably a pressure vessel, is designed to hold mixtures of carbon dioxide/cleaning solvent/residues which have been conveyed from the cleaning vessel 10 from the cleaning steps. This vessel 20 is designed to hold the pressurized cleaning solvent and to accommodate pressure reduction by venting off carbon dioxide, or de-carbonating, from the cleaning solvent. A detergent additive system 22 is designed to store and then convey various mixtures of detergents and additives to the cleaning vessel. The cleaning solvent holding tank 26 is designed to store pressurized cleaning solvent from the cleaning vessel 10. A cleaning solvent still 28, preferably a vacuum distillation system, is designed to efficiently distill and purify the cleaning solvent by separating residues from the solvent. Filters 30 and mist eliminators 32 are designed to collect particulate residues and aerosols as fluids pass through them. A gaseous carbon dioxide compressor 34 is provided to move carbon dioxide from one tank to another, typically from still to storage tank, or from cleaning vessel to storage, or from storage tank to cleaning vessel. Finally, a liquid carbon dioxide pump 36 is used to convey liquid carbon dioxide from the storage tank to cleaning vessel.

Although the present invention has been described with reference to preferred embodiments, workers skilled in the art will recognize that changes may be made in form and detail without departing from the spirit and scope of the invention.

1. A method for cleaning and decontaminating apparel comprising:
providing a pressurizable cleaning vessel;
providing a permeable container;
placing the apparel within the permeable container;
placing the permeable container containing the apparel within the pressurizable cleaning vessel;
filling the cleaning vessel with a solvent, the solvent being a mixture selected from at least one of propylene glycol ether, water, organic acid or carbon dioxide;
allowing the permeable container and the apparel to be in contact with the solvent for a first selected period of time;
after contacting the permeable container and apparel with the solvent for the first selected period of time, introducing into the pressurizable cleaning vessel carbon dioxide gas under pressure to carbonate the solvent;
allowing the permeable container and the apparel to be in contact with the carbonated solvent for a second selected period of time;
after the permeable container and the apparel has been in contact with the carbonated solvent for the second selected period, removing at least a portion of the solvent from the cleaning vessel while under pressure;
introducing into the pressurizable cleaning vessel a rinsing solution consisting essentially of liquid carbon dioxide and alcohol;
agitating the cleaning vessel containing the rinsing solution, while under pressure, for a third selected period of time;
removing the rinsing solution from the pressurizable cleaning vessel while under pressure;
depressurizing the cleaning vessel; and
removing the permeable container containing the apparel from the pressurizable cleaning vessel.

2. The method of claim 1, wherein the apparel includes personal protective equipment.

3. The method of claim 2, wherein the personal protective equipment includes clothing, helmets, goggles, pants, coats, gloves, hoods, boots and interface elements.

4. The method of claim 1 whereupon contacting the permeable container and apparel with the solvent for the first selected period of time, the introduction into the pressurizable cleaning vessel of the carbon dioxide gas is done between 200 to 1000 psig.

5. The method of claim 4, further comprising decreasing the pressure of the cleaning vessel to at least 200 psig, and then increasing the pressure up to 800 psig.

6. The process of claim 5, further comprising repeating the cycle of decreasing and increasing the pressure for up to 10 cycles.

7. The process of claim 1 wherein agitating the cleaning vessel containing the rinsing solution occurs at pressures between 250 to 1000 psig.

8. The process of claim 7 wherein agitating the cleaning vessel containing the rinsing solution occurs at pressures between 400 and 700 psig.
